(19) United States
(12) Patent Application Publication
(10) Pub. No.: US 2023/0022158 A1
(43) Pub. Date: Jan. 26, 2023

(54) PRODUCT PACKAGING HAVING SELF-STERILIZING COMPONENTS

(71) Applicant: AMCOR FLEXIBLES NORTH AMERICA, INC., Neenah, WI (US)

(72) Inventors: Jerome E. McGinnis, Neenah, WI (US); Tyler C. Yager, Parkers Prairie, WI (US)

(21) Appl. No.: 17/783,719

(22) PCT Filed: Dec. 31, 2019
(86) PCT No.: PCT/US19/69114

§ 371 Date: Jun. 9, 2022

(51) Int. Cl. B65D 81/24; B65D 81/20; B32B 27/34; B32B 27/32; B32B 27/08; A61B 50/30; A61L 2/26; A61L 2/20

(52) U.S. Cl. CPC B65D 81/24; B65D 81/2084; B32B 27/34; B32B 27/32; B32B 27/08; A61B 50/30; A61L 2/26; A61L 2/20; B32B 2307/518; B32B 2439/46; B32B 2255/10; B32B 2255/26; B32B 2439/70; B32B 2439/80; B32B 2307/7244; B32B 2553/00; B32B 2307/7246; B32B 2307/31; A61B 2050/314; A61L 2101/06

(57) ABSTRACT

A package comprises: a sterile barrier packaging film; self-sterilizing components comprising: a plurality of chlorite ions and water, wherein the self-sterilizing components are substantially free of an energy-activated catalyst and of an acid-releasing compound; a package interior formed by hermetically sealing the sterile barrier packaging film; and a headspace within the package interior comprising carbon dioxide present in an amount of greater than or equal to 5% by volume of the headspace. When the package is exposed to ultraviolet (UV) light having a wavelength of 254 nm, the chlorite ions react with the water to generate chlorine dioxide (ClO₂), which is released into the headspace. In the absence of any UV light, there is not chlorine dioxide (ClO₂) generation.

### TECHNICAL FIELD

The present disclosure relates generally to packages for various products that generate a disinfectant gas. In particular, packages are self-sterilizing by providing controlled-release of chlorine dioxide (ClO₂) gas for sterilizing various products including food, medical devices, and medical supplies.

### BACKGROUND

Chlorine dioxide (ClO₂) is a powerful oxidizing agent and disinfectant. Packages that generate ClO₂ and are self-sterilizing can be cheaper, safer, and quicker alternatives to other sterilization methods such as exposing products to ethylene oxide (EtO) gas. Timing and amount of release of ClO₂ gas in packaging, however, can be difficult to control.

Some technologies rely on chlorate-based reactions in the presence of a co-reactant. In U.S. Appln. Pub. No. 2018/272019A1, a chlorite or chlorate salt is used with co-reactants that are an oxidant and/or an acid. Some technologies rely on a different chemical reaction involving chlorite ions, water, and ultra-violet light, which does not utilize an energy-activated catalyst or an acid-releasing compound. In WO2017/031349A1, a chlorine dioxide-producing layer includes a polymer composition and a plurality of chlorite ions. The chlorine dioxide-producing layer is substantially free of an energy-activated catalyst and is substantially free of an acid-releasing compound. However, the film is capable of generating chlorine dioxide when exposed to UV light and moisture.

There is a need for packages that release ClO₂ gas for deodorizing, disinfecting, and/or sterilizing in a manner that is controlled and efficient.

### SUMMARY

Provided are packages that release ClO₂ gas from reaction of self-sterilizing components for deodorizing, disinfecting, and/or sterilizing in a manner that is controlled and efficient.

An aspect is a package comprising: a sterile barrier packaging film; self-sterilizing components comprising: a plurality of chlorite ions and water, wherein the self-sterilizing components are substantially free of an energy-activated catalyst and of an acid-releasing compound; a package interior formed by hermetically sealing the sterile barrier packaging film; and a headspace within the package interior comprising carbon dioxide present in an amount of greater than or equal to 5% by volume of the headspace. When the package is exposed to ultraviolet (UV) light having a wavelength of 254 nm, the chlorite ions react with the water to generate chlorine dioxide (ClO₂), which is released into the headspace. In the absence of any UV light, there is not chlorine dioxide (ClO₂) generation. The water in the package is proximate to the plurality of chlorite ions.

In one or more embodiments, a relative humidity in the package interior is in the range of greater than or equal to 20% to less than or equal to 100% at 72.5° F.

In one or more embodiments, a salt comprising sodium chlorite, potassium chlorite, calcium chlorite, magnesium chlorite, lithium chlorite, ammonium chlorite, or mixtures thereof supplies the plurality of chlorite ions.

In one or more embodiments, the sterile barrier packaging film is substantially ultraviolet (UV)-light transparent.

In one or more embodiments, the sterile barrier packaging film comprises a primary layer and a patch affixed to the primary layer, wherein the patch supplies the plurality of chlorite ions. In an embodiment, the patch comprises: a patch support that is permeable to chlorine dioxide and that defines a first major surface of the patch; and a patch sealing layer comprising the plurality of chlorite ions dispersed in a polymer composition, wherein the patch sealing layer is in contact with the patch support and defines a second major surface of the patch, and wherein the patch sealing layer is affixed to the primary layer.

In one or more embodiments, the plurality of chlorite ions is present in a layer of the self-sterilizing packaging film, and a salt in combination with a polymer composition supplies the plurality of chlorite ions.

In an embodiment, the polymer composition comprises at least one of a polyethylene, a polypropylene, a polyacrylate, or a copolymer of any of these.

Another aspect is packaged product comprising: a packaging film; a plurality of chlorite ions; water; a package interior formed by hermetically sealing the packaging film; a product within the package interior; and a headspace within the package interior comprising carbon dioxide present in an amount of greater than or equal to 5% by volume of the headspace; wherein self-sterilizing components comprise: the water and the plurality of chlorite ions, and are substantially free of energy-activated catalyst and are substantially free of an acid releasing compound.

In an embodiment, when the film is exposed to ultraviolet (UV) light having a wavelength of 254 nm, reaction of the chlorite ions with the water generates chlorine dioxide (ClO₂), which is released into the headspace and is effective to sterilize the product within the package interior.

In an embodiment, the packaging film comprises a primary layer and a patch affixed to the primary layer, wherein the patch supplies the plurality of chlorite ions. In an embodiment, the patch comprises: a patch support that is permeable to chlorine dioxide; and a patch sealing layer comprising the plurality of chlorite ions dispersed in a polymer composition, wherein the patch sealing layer is in contact with the patch support, and wherein the patch sealing layer is affixed to the primary layer. In an embodiment, the polymer composition comprises at least one of: a polyethylene, a polypropylene, a polyacrylate, or a copolymer of any of these.

The product within the package interior may be a medical device or a medical supply. The product within the package interior may be a food.

A further aspect is a method of sterilizing a packaged product comprising: providing an unsealed package comprising: a sterile barrier packaging film comprising a primary layer and a plurality of chlorite ions; and a package interior; and introducing water to the package interior; introducing a product to be sterilized to the package interior, introducing a gas comprising carbon dioxide to the package interior of the package; hermetically sealing an open end of the unsealed package after introducing the gas comprising carbon dioxide to form a self-sterilizing package containing a packaged product and a headspace, wherein the headspace comprises the carbon dioxide in an amount of greater than or equal to 5% by volume of the headspace; and exposing the self-sterilizing package to UV radiation; wherein self-sterilizing components comprise: the water and the plurality of chlorite ions, and are substantially free of an energy-activated catalyst and of an acid-releasing compound. The water is proximate to the plurality of chlorite ions.

In one or more embodiments, an amount of at least 1.39 micrograms/milliliter of chlorine dioxide is generated within the package within a timeframe of 5 minutes.

In one or more embodiments, the UV radiation includes a 254 nm wavelength.

These and other aspects of the invention are described in the detailed description below. In no event should the above summary be construed as a limitation on the claimed subject matter.

### BRIEF DESCRIPTION OF THE DRAWINGS

The disclosure may be more completely understood in consideration of the following detailed description of various embodiments of the disclosure in connection with the accompanying drawings, in which:

FIG. 1 is a schematic side view of a sterile barrier packaging film according to an embodiment;

FIG. 2 is a schematic side view of a sterile barrier packaging film according to an embodiment;

FIG. 3 is a schematic plan view of a package according to an embodiment; and

FIG. 4 is a schematic sectional view of a package according to an embodiment.

The figures are not necessarily to scale. Like numbers used in the figures refer to like components. It will be understood, however, that the use of a number to refer to a component in a given figure is not intended to limit the component in another figure labeled with the same number.

### DETAILED DESCRIPTION

Packages of the present disclosure comprise a sterile barrier packaging film; self-sterilizing components comprising: a plurality of chlorite ions and water, wherein the self-sterilizing components are substantially free of an energy-activated catalyst and of an acid-releasing compound; a package interior formed by hermetically sealing the sterile barrier packaging film; and a headspace within the package interior comprising carbon dioxide (CO₂) present in an amount of greater than or equal to 5% by volume of the headspace. When the package is exposed to ultraviolet (UV) light having a wavelength of 254 nm, the chlorite ions react with the water to generate chlorine dioxide (ClO₂), which is released into the headspace. In the absence of any UV light, there is not chlorine dioxide (ClO₂) generation. Water in the package is proximate to the chlorite ions.

Some currently available packages that generate ClO₂ and are self-sterilizing pose problems in that timing and amount of release of ClO₂ gas in packaging can be difficult to control. The packages and methods herein provide a solution to such problems by releasing ClO₂ gas for deodorizing, disinfecting, and/or sterilizing in a manner that is controlled and efficient. This is accomplished by including CO₂ gas in the headspace of the packages. One way efficiency is achieved is due to the yield of ClO₂ gas generated for any given chlorite ion concentration and delivery format is increased as compared to a headspace without added CO₂ gas. One way control is achieved is the use of a reaction scheme that relies on UV light, discussed in the following with respect to reaction (1). The combination of including CO₂ gas in the headspace of the packages with generating ClO₂ gas by way of a UV-activated mechanism has advantageously resulted in providing self-sterilizing packages whose efficacy is increased by way of headspace management. For both medical device and food packages, the present disclosure offers a low-cost solution with minimal changes to existing packaging lines and procedures to achieve benefits in control and/or efficiency.

Using CO₂ gas in the headspace of packages is shown herein to significantly improve the yield of ClO₂, on the order of 1.5 times and higher concentrations relative to air only, by way of the reaction according to the following:

3ClO₂⁻H₂O+hv⇒Cl³¹ +2ClO₂+2OH⁻+0.5O₂ (1).

Without intending to be bound by theory, it is understood that CO₂ dissolves in water that is added in a moisturization step to a chlorite ion-containing package. The dissolution results in the formation of carbonic acid. The weak acid offsets the base (i.e. hydroxyl, OH—), helping to drive the reaction according to (1) to the right. Ultimately, the carbonic acid lowers the pH enough to significantly increase the yield of the reaction in the presence of UV light according to (1). It is noted that (1) excludes the presence of an energy-activated catalyst and an acid-releasing compound. The presence of CO₂ and formation of carbonic acid (HCO₃), however, does not create an acidic enough environment to induce a spontaneous generation of ClO₂ gas from the chlorite ions present. Therefore, generation of ClO₂ gas is still controlled by UV light activation (hv)). The packaging described herein releases ClO₂ gas on-demand upon exposure to ultraviolet (UV) light and the addition of CO₂ to the package has the effect of increasing the ClO₂ gas generation achieved as compared to a headspace without CO₂.

The amount of chlorite ions present in the package is effective to deodorize, disinfect, and/or sterilize a product disposed in the interior of the package upon exposure to ultraviolet (UV) light having a wavelength of 254 nm. The concentration of chlorite ion in, for example, a layer of the package or a coating on a layer of a package or a patch affixed to a layer of the package may be varied in amount to deliver a desired amount of ClO₂ gas. Also, the concentration of chlorite ion may be varied depending on the thickness of the layer or coating or patch, the surface area (length×width) and/or patterning of the layer or coating or patch, the volume of the interior of the package, and the effect desired (such as deodorize, disinfect, or sterilize). Any suitable amount of chlorite ion may be included to deodorize, disinfect, or sterilize the interior of a package and the contents of the package may vary depending on the volume of the interior of the package. Advantageously, by including CO₂ gas in the headspace of the packages herein, the yield of ClO₂ gas generated for any given chlorite ion concentration and delivery format is increased as compared to a headspace without CO₂ gas. In this way, chlorite ions are used more efficiently. For example, when CO₂ gas is present, a lower amount of chlorite ions would be needed to achieve the same amount of ClO₂ gas as compared to the absence of CO₂ gas in the headspace.

Reference to “substantially transparent to ultraviolet radiation” means that transmission of UV light through a layer or film is at least enough to allow the reaction of (1) to occur. In one or more embodiments, at least 10% of ultraviolet light can be transmitted through the layer or film.

As used in this disclosure, water being “proximate” to the chlorite ions means close enough to dissociate a salt that is supplying the ions. In one or more embodiments, the water is in contact with the chlorite ions.

In one or more embodiments, ClO₂ gas concentration in the headspace is measured by extracting a gaseous sample of the headspace and dissolving the ClO₂ in water, which is then tested by traditional technologies that measure ClO₂ concentration in water. An exemplary technology is conducted on an apparatus called ChlordioX Plus supplied by Palintest.

In one or more embodiments, CO₂ gas concentration in the headspace is measured by traditional gas detectors suitable for detecting CO₂. An exemplary technology is conducted on an apparatus called Dansensor® supplied by Mocon or an apparatus called PAC CHECK also supplied by Mocon.

### Package

Materials suitable for making hermetically sealed packages are used herein. Hermetically sealed packages herein are effective to retain ClO₂ for a duration that facilitates one or more of deodorizing, disinfecting, and sterilization in the package. Packages comprise a sterile barrier packaging film comprising a primary layer and self-sterilizing components.

Any suitable package may include a layer or coating comprising chlorite ions.

Any suitable package may include a patch that comprises a patch support that is permeable to chlorine dioxide and a sealing layer comprising chlorite ions.

At least a portion of the package is substantially transparent to ultraviolet light. The package may be in the form of a bag, pouch, or other suitable container.

In one or more embodiments, packages herein are in accordance with ISO 11607-1 (2006), “Packaging for terminally sterilized medical devices”.

Preferably, packages and correspondingly self-sterilizing components described herein release an amount of chlorine dioxide in a sufficient amount to deodorize or disinfect or sterilize a product in the package. The chlorine dioxide gas is contained within the package for a sufficient amount of time to deodorize or disinfect or sterilize the product in the package. The chlorine dioxide gas is generally generated over a time period of UV light exposure, which may be relatively short, for example, on the order of seconds or minutes. Subsequent deodorizing or disinfecting or sterilizing occurs while the gas is contained, which may be a relatively longer time, for example, on the order of hours or days. In one or more embodiments, the packages herein are effective to disinfect and/or sterilize a medical device. In one or more embodiments, the packages herein are effective to deodorize and/or disinfect food products.

Suitable products include but are not limited to medical devices, medical supplies, and food.

Reference to “medical device” includes but is not limited to a diagnostic tool, an implant, a surgical tool, and a medical instrument. Reference to “medical supplies” includes but is not limited to bandages, gauze, tubing, catheters, and syringes.

As used herein, “deodorize” means to remove or conceal an unpleasant smell. In many cases, the unpleasant smell may be caused by odor-causing bacteria and killing of the bacteria may have a deodorizing effect. A package and correspondingly the self-sterilizing components described herein may release any suitable amount of ClO₂ gas to deodorize an article stored in the package, such as a food product, which may be, for example, produce. For example, at least 2 parts per million (ppm) ClO₂ may be released into a package interior. Typically, release of at least 10 ppm ClO₂ gas is enough to deodorize produce. The concentration of chlorine dioxide may increase over time if the package is sealed, as additional chlorine dioxide is released. The amount of ClO₂ gas needed to effectively deodorize an article stored in the package will depend, in part, on the nature of the article. In addition, the time that the article is exposed to ClO₂ gas will affect the ability of the ClO₂ gas to deodorize the article. In some embodiments, an amount of ClO₂ gas is released for a time sufficient to expose the article to at least 2 ppm-hours of ClO₂ gas to deodorize the article. For example, an amount of chlorine dioxide to result in at least 10 ppm-hours of ClO₂ gas, or at least 20 ppm-hours of ClO₂ gas may be released to deodorize the article.

As used herein, “disinfect” means to reduce the number of living bacteria. To determine whether a product is disinfected, a product that has undergone a disinfecting treatment, such as exposure to ClO₂ gas, can be compared to a control product that has not undergone the disinfecting treatment to determine whether bacterial burden has been reduced; and, if so, the product will be considered to have been disinfected. Alternatively, the bacterial burden of a product may be compared before and after treatment to determine whether the product has been disinfected. A package and correspondingly the self-sterilizing components described herein may release any suitable amount of ClO₂ gas to disinfect a medical device disposed within package. For example, 10 parts per million (ppm) or greater ClO₂ gas may be released into a package interior. Typically, the self-sterilizing components may release 50 ppm or greater ClO₂ gas to disinfect the medical device. The concentration of chlorine dioxide may increase over time if the package is sealed, as additional chlorine dioxide is released. The amount of ClO₂ gas needed to effectively disinfect a medical device will depend, in part, on the nature of the device. In addition, the time that the medical device is exposed to ClO₂ gas will affect the ability of the ClO₂ gas to disinfect the medical device. In some embodiments, the self-sterilizing components release an amount of ClO₂ gas for a time sufficient to expose the medical device to 100 ppm-hours or greater of ClO₂ gas to disinfect the product, including 150 ppm-hours or more of ClO₂ gas, or 200 ppm-hours or more of ClO₂ gas, to disinfect the medical device.

As used herein, “sterilize” means to make free from bacteria or other living organisms. A package and correspondingly the self-sterilizing components described herein may release any suitable amount of ClO₂ gas to sterilize a medical device disposed within the package. For example, 200 parts per million (ppm) or greater ClO₂ gas may be released into a package interior. Typically, the self-sterilizing components may release 500 ppm or greater ClO₂ gas to sterilize the medical device. The amount of ClO₂ gas needed to effectively sterilize a medical device will depend, in part, on the nature of the device. In addition, the time that the medical device is exposed to ClO₂ gas will affect the ability of the ClO₂ gas to sterilize the device. In some embodiments, the self-sterilizing components release an amount of ClO₂ gas for a time sufficient to expose the medical device to 1000 ppm-hours or greater of ClO₂ gas to sterilize the device, including 1500 ppm-hours or more of ClO₂ gas, or 2000 ppm-hours or more of ClO₂ gas, to sterilize the medical device.

### Self-Sterilizing Components

The self-sterilizing components comprise water and a plurality of chlorite ions, wherein the self-sterilizing components are substantially free of an energy-activated catalyst and of an acid-releasing compound. Reference to “self-sterilizing components” means those compounds participating in the reaction to make ClO₂ gas.

hi one or more embodiments, the self-sterilizing components consist essentially of water and a plurality of chlorite ions to the exclusion of an energy-activated catalyst and an acid-releasing compound. As used herein, an “acid-releasing compound” is a compound that, in the presence of moisture, can generate acid and hydronium ions, which hydronium ions can react with chlorite ions to form ClO₂ gas. U.S. Pat. No. 6,605,304 lists a number of acid releasing compounds for gas generation including carboxylic acids, esters, anhydrides, acyl halides, phosphoric acid, phosphate esters, trialkylsilyl phosphate esters, dialkyl phosphates, sulfonic acid, sulfonic acid esters, sulfonic acid chlorides, phosphosilicates, phosphosilicic anhydrides, carboxylates of poly a-hydroxy alcohols such as sorbitan monostearate or sorbitol monostearate, phosphosiloxanes, and acid releasing waxes, such as propylene glycol monostearate acid releasing waxes. U.S. Pat. No. 6,605,304 also lists as acid-releasing compounds inorganic acid releasing agents, such as polyphosphates, including tetraalkyl ammonium polyphosphates, monobasic potassium phosphate, potassium polymetaphosphate, sodium metaphosphates, borophosphates, aluminophosphates, silicophosphates, sodium polyphosphates such as sodium tripolyphosphate, potassium tripolyphosphate, sodium-potassium phosphate, and salts containing hydrolysable metal cations such as zinc. In some embodiments described herein, the chlorine dioxide-producing layers or the films for generating ClO₂ gas described herein are substantially-free of such compounds. In one or more embodiments, the self-sterilizing components exclude carbonic acid.

Any suitable source of chlorite ion may be used. Typically, the source of chlorite ion is a chlorite salt. A “chlorite salt” as used herein is not limited to embodiments wherein the anion and cation form a solid crystal, but in fact encompass any form in which such salts are known to exist, including in aqueous or other solutions or dispersed within a polymeric matrix. In some embodiments, the cation of the chlorite salt is an organic cation, and in some embodiments the cation of the chlorate salt is inorganic. In some such embodiments, the chlorite salt is sodium chlorite, potassium chlorite, calcium chlorite, magnesium chlorite, lithium chlorite or ammonium chlorite. In some embodiments, the chlorite salt is sodium chlorite.

As used herein, an “energy-activated catalyst” is a compound that can catalyze the oxidation of ClO⁻ to ClO₂ gas following activation of the catalyst compound by electromagnetic energy, such as visible light.

As used herein, “substantially free of an energy-activated catalyst” means that the self-sterilizing components include no energy-activated catalyst or includes an amount of an energy-activated catalyst that does not significantly contribute to ClO₂ gas generation. In some embodiments, the ratio (by weight) of an energy-activated catalyst to chlorite ion source, such as chlorite ion salt, in the chlorine dioxide-producing layer is less than or equal to 1:2. For example, the ratio of energy-activated catalyst to chlorite ion source may be less than or equal to 1:5, such as less than or equal to 1:10, or less than or equal to 1:20.

As used herein, an “acid-releasing compound” is a compound that, in the presence of moisture and the absence of UV light, can generate acid and hydronium ions, which hydronium ions can react with chlorite ions to form ClO₂ gas. Herein, although CO₂ is present and its dissolution into water can create carbonic acid, the carbonic acid does not participate in the reaction according to (1) and therefore the self-sterilizing components do not include carbonic acid.

As used herein, “substantially free of an acid-releasing compound” means that the self-sterilizing components include no acid-releasing compound or includes an amount of an acid-releasing compound that does not significantly contribute to ClO₂ gas generation. In some embodiments, the ratio (by weight) of acid-releasing compound to chlorite ion source, such as chlorite ion salt, in the chlorine dioxide-producing layer is less than or equal to 1:10. For example, the ratio of acid releasing compound to chlorite ion source may be less than or equal to 1:20, such as less than or equal to 1:50, or less than or equal to 1:100.

Water is present to participate in the reaction according to (1). In one or more embodiments, relative humidity in the package at 72.5° F. is greater than or equal to 20% to less than or equal to 100% and all values and subranges therebetween, including but not limited to greater than or equal to 40% to less than or equal to 100%, or preferably greater than or equal to 60% to less than or equal to 100%.

In one or more embodiments, the plurality of chlorite ions may be supplied in a layer of the self-sterilizing packaging film, by a salt in combination with a polymer composition.

In one or more embodiments, a patch supplies the plurality of chlorite ions.

### Sterile Barrier Packaging Film

The sterile barrier packaging film is a barrier that is effective to prevent transmission of microbes. In one or more embodiments, the sterile barrier packaging film comprises a primary layer and self-sterilizing components. At least a portion of the sterile barrier packaging film is substantially ultraviolet (UV)-light transparent. For example, one sidewall of the package may be transparent and the other may not be. The transparency is adequate to transmit UV light through the package and permitting the UV light to contact the self-sterilizing components when the package is hermetically sealed.

The sterile barrier packaging film may comprise a single or multilayer film. The sterile barrier packaging film may be flexible or rigid, depending on the type of package being formed. The sterile barrier packaging film may be, for example, a side of a bag, pouch or container, or may be a lid for a container such as a thermoformed tray.

In one or more embodiments, the sterile barrier packaging film comprises a primary layer. In an embodiment, the sterile barrier packaging film comprises the primary layer and a patch affixed to the primary layer, wherein the patch supplies the plurality of chlorite ions. In an embodiment, the primary layer of the sterile barrier packaging film comprises the plurality of chlorite ions and a polymer composition.

In another embodiment, the plurality of chlorite ions is present in a coating on the self-sterilizing packaging film. The coating comprises a chlorite salt in combination with a polymer composition. Any suitable coating composition may be used to coat the self-sterilizing packaging film. For example, the coating composition may comprise one or more chlorite salts, one or more other suitable coating components, and one or more suitable solvents or diluents. In some embodiments, the one or more coating components are water soluble or water dispersible.

Suitable coating components may include materials that retain the chlorite ions on the self-sterilizing packaging film. In some embodiments, the coating composition comprises a polymer or resin compatible with the self-sterilizing packaging film to be coated. Upon drying or curing of the coating, the coating preferably adheres to the self-sterilizing packaging film.

The coating composition may comprise any suitable polymer. In some embodiments, the coating composition comprises one or more of polyethylene, ethylene vinyl acetate, ethylene alpha-olefins, or polypropylene.

The coating compositions may include any suitable amount of chlorite ion, such as the amounts discussed above. In some embodiments, the chlorite ions are present in a salt, and the salt is present in an amount within a range from 0.1 weight percent to 30 weight percent relative to the total weight of the coating. For example, the salt may present in an amount within a range from 10 weight percent to 20 weight percent relative to the total weight of the coating.

The coating composition may be applied in any suitable manner. For example, the surface to be coated may be dipped in the coating composition or the coating composition may be sprayed, rolled, printed, or otherwise deposited on the surface of the film. In some embodiments, the coating is pattern coated to coat certain portions of a surface of the self-sterilizing packaging film and to leave certain portions of the self-sterilizing packaging film uncoated.

In another embodiment, the plurality of chlorite ions is present in a layer of the self-sterilizing packaging film. That is, a chlorite salt is incorporated into one of the layers of the self-sterilizing packaging film.

In one or more embodiments, the primary layer of the sterile barrier packaging film is a sealing layer, which seals to form a hermetic seal. That is, the sealing layer comprises a thermoplastic polymer or polymer mixture that softens when exposed to heat and returns to its original condition when cooled to room temperature. The sealing layer could be used to bond the sterile barrier packaging film to a base material to form a peripheral seal, normally by heat sealing. The base material may be a rigid or flexible thermoformed tray. The base material may be a film of the same type or of a different type as the sterile barrier packaging film. In general, the sealing layer may comprise any suitable polymer composition or thermoplastic material including, but not limited to, synthetic polymers such as polyesters, polyamides, polyolefins, polystyrenes, and the like. Thermoplastic materials may also include any synthetic polymers that are cross-linked by either radiation or chemical reaction during a manufacturing or post-manufacturing process operation. Exemplary polyolefins include polyethylene (PE) and polypropylene (PP).

In one or more embodiments, the primary layer and/or one or more further layers provide barrier functionality comprising a gas barrier and/or a moisture barrier.

A gas barrier layer is preferably an oxygen barrier layer and is preferably a core layer positioned between and protected by surface layers. For example, an oxygen barrier layer can be in contact with a first surface layer and an adhesive layer or may be sandwiched between two tie layers, two surface layers, or a tie layer and a surface layer.

A gas barrier, such as a chlorine dioxide barrier or an oxygen barrier, is preferably selected to provide sufficiently diminished permeability of gases to protect a product disposed in the sealed packaging from undesirable deterioration or, for example, oxidative processes. For example, a film may comprise an oxygen barrier having an oxygen permeability that is low enough to prevent or slow oxidation of products to be packaged in the film. In some embodiments, the packaging film has an oxygen transmission rate (O₂TR) of less than 150 cm³/m²/24 hours at 1 atmosphere and 23° C., such as less than 10 cm³/m² per 24 hours at 1 atmosphere and 23° C. To protect oxygen sensitive articles from deterioration from oxygen contact over time, the films may have an O₂TR of less than 1, such as less than 0.1, less than 0.01, or less than 0.001 cm³/m² per 24 hours at 1 atmosphere and 23° C.

A moisture barrier is preferably selected to provide a moisture permeability sufficiently diminished to protect an article disposed in the sealed packaging from undesirable deterioration. For example, a film may comprise a water barrier having a moisture permeability that is low enough to prevent deleterious effects upon packaged articles such as medical devices. A preferred film according to various embodiments will have a water vapor transmission rate (WVTR) of less than 15 g/m² per 24 hours at 38° C. and 90% RH. In some embodiments, a film has a WVTR of less than 1, less than 0.1, or less than 0.01 g/m² per 24 hours at 38° C. and 90% RH.

A barrier layer can comprise any suitable material and may be any suitable thickness. A gas barrier layer can comprise polyvinyl alcohol (PVOH), ethylene vinyl alcohol (EVOH), polyvinylidene chloride (PVDC), polyamide, polyester, polyalkylene carbonate, polyacrylonitrile, a nanocomposite, a metallized film such as aluminum vapor deposited on a polyolefin, etc., as known to those of skill in the art. Suitable moisture barrier layers include aluminum foil, PVDC, fluoropolymers like polychlorotrifluoroethylene (PCTFE), polyolefins such as HDPE, LLDPE and cyclic olefin copolymers (COC), and metallized films such as aluminum vapor deposited on a polyolefin, etc., as known to those of skill in the art. It is desirable that the thicknesses of the barrier layers be selected to provide the desired combination of the performance properties sought e.g. with respect to oxygen permeability, water vapor permeability, delamination resistance, etc.

If a film comprises a moisture barrier, care may need to be taken to ensure that the chlorite ions (e.g., a chlorite ion-containing sealing layer or coating layer) of the film is capable of being exposed to sufficient moisture to release ClO₂ gas. In some embodiments, the atmosphere of the packaging manufacturing line can be controlled to ensure that the chlorite-containing layer is exposed to sufficient moisture. In some embodiments, the packaging may be in the form of a three-side sealed bag with the article (e.g., food product, pharmaceutical product, medical device, or other product) disposed in the bag prior to final sealing of the fourth side to seal the product in the bag. While the product is in the three-side sealed bag, moist gas such as a stream of nitrogen containing steam or heated water may be used to flush the bag and to provide sufficient moisture for generation of ClO₂ gas prior to final sealing. In some embodiments, the packaging films may be stored in a high moisture environment prior to being brought on-line for packaging.

The sterile barrier packaging film optionally further comprises a secondary layer that may serve as an outer layer of the package. An outer layer is seen by a user/consumer and is generally exposed to the environment surrounding the package. In both monolayer and multilayer embodiments, an exterior surface of the film may have desirable appearance properties and may have high gloss. Also, it preferably withstands contact with sharp objects and provides abrasion resistance, and for these reasons the outer layer is often termed the abuse resistant layer. This exterior abuse-resistant layer may or may not also be used as a heat sealable layer and thus may comprise one or more suitable heat seal polymers such as polyethylene or polypropylene. As the exterior surface layer of the film, this layer most often is also the exterior layer of any package, bag, pouch or other container made from the film, and is therefore subject to handling and abuse e.g. from equipment during packaging, and from rubbing against other packages and shipping containers and storage shelves during transport and storage.

The exterior surface layer should be easy to machine (i.e. be easy to feed through and be manipulated by machines e.g. for conveying, packaging, printing or as part of the film or bag manufacturing process). Suitable stiffness, flexibility, flex crack resistance, modulus, tensile strength, coefficient of friction, printability, and optical properties are also frequently designed into exterior layers by suitable choice of materials. This layer may also be chosen to have characteristics suitable for creating desired heat seals which may be resistance to burn through e.g. by impulse sealers or may be used as a heat-sealing surface in certain package embodiments e.g. using overlap seals.

Suitable exterior surface layers may comprise polyamide, polyethylene, polypropylene, or copolymers, or blends thereof. Oriented films of this or any other layer may be either uni-axially or bi-axially oriented. The exterior layer thickness is typically 0.5 to 2.0 mils. Thinner layers may be less effective for abuse resistance, however thicker layers, though more expensive, may advantageously be used to produce films having unique highly desirable puncture resistance and/or abuse resistance properties.

In some embodiments, the outer layer is transparent to UV light.

The sterile harrier packaging film optionally further comprises other layers including but not limited to bulk layers, intermediate, tie layers, and reflective layers.

A bulk layer may be provided to provide additional functionality such as stiffness or heat sealability or to improve machinability, cost, flexibility, barrier properties, etc. The bulk layer may be of any suitable thickness or may even be omitted for use in certain applications.

A film as described herein may comprise one or more adhesive layers, also known in the art as “tie layers,” which can be selected to promote the adherence of adjacent layers to one another in a multilayer film and prevent undesirable delamination.

A film herein can comprise any suitable number of tie or adhesive layers of any suitable composition. Various adhesive layers are formulated and positioned to provide a desired level of adhesive between specific layers of the film according to the composition of the layers contacted by the tie layers.

The interior, exterior, intermediate or tie layers may be formed of any suitable thermoplastic materials, for example, polyamides, polystyrenes, styrenic copolymers e.g. styrene-butadiene copolymer, polyolefins, and in particular members of the polyethylene family such as LLDPE, VLDPE, HDPE, LDPE, COC, ethylene vinyl ester copolymer or ethylene alkyl acrylate copolymer, polypropylenes, ethylene-propylene copolymers, ionomers, polybutylenes, alpha-olefin polymers, polyesters, polyurethanes, polyacrylamides, anhydride-modified polymers, acrylate-modified polymers, polylactic acid polymers, or various blends of two or more of these materials.

Various additives may be included in the polymers utilized in one or more of the primary, secondary, base, coating, exterior, interior, and intermediate or tie layers of packaging comprising the same. For example, a layer may be coated with an anti-block powder. Also, conventional antioxidants, antiblock additives, polymeric plasticizers, acid, moisture or gas (such as oxygen) scavengers, slip agents, colorants, dyes, pigments, organoleptic agents may be added to one or more film layers of the film or it may be free from such added ingredients.

The packaging films may include one of more layers that reflect UV light. Examples of suitable materials for such layers include metallic foils or depositions like vacuum metallized or sputtered layers. The reflective layer could be applied as a coating where reflective particles such as metallic flakes are dispersed in a polymeric binder. The film may be configured such that the self-sterilizing components are positioned between the reflective layer and the UV source when the film is exposed to UV radiation. In some such embodiments, the one or more reflective layer(s) is/are in contact with a layer of the film. The reflective layers may be optically engineered to maximize yield, by increasing UV exposure of the chlorite salts dispersed within the film (e.g., dispersed within a sealing layer or a coating disposed on the sealing layer).

Sterile barrier packaging films described herein may have any suitable thicknesses. In some embodiments, the packaging film has a total thickness of less than about 50 mils, more preferably the film has a total thickness of from about 1.0 to 10 mils (25-250 microns (μ), such as from about 1 to 5 mils, or from about 2 to 3.5 mils. For example, entire multilayer films or any single layer of a multilayer film can have any suitable thicknesses, including 1, 2, 3, 4, 5, 6, 7, 8, 9, 10 or 50 mils, or any increment of 0.1 or 0.01 mil therebetween.

In some embodiments, the packaging films are as thick as 50 mils (1270 microns) or higher, or as thin as 1 mil (25.4 microns) or less. In various embodiments, the packaging films have a thickness of between about 2-4 mil (51-102 microns).

### Patches

The patches comprise a patch support permeable to chlorine dioxide and a patch sealing layer. The patch support defines a first major surface and the patch sealing layer defines a second major surface of the patch. In one or more embodiments, the patch is according to WO2019/132934A1, to common assignee, the disclosure of which is incorporated herein by reference in its entirety.

The patch may be affixed to an inner surface of the sterile barrier packaging film.

A film that forms the patch or sheet may comprise, consist essentially of, or consist of the patch support and the sealing layer comprising the chlorite ions. The patch support may comprise one or more layers, provided that chlorine dioxide is permeable through each layer.

The film may be in the folio of a sheet from which one or more patches may be formed. For example, the sheet may be punched or cut to form the patches. As used herein, a “sheet” includes a roll of the film.

The patch support may be formed from material that is permeable to chlorine dioxide or may be modified to be permeable to chlorine dioxide. For example, any support that is not initially permeable to chlorine dioxide may be made permeable by perforating to form a patch support. Accordingly, a wide variety of materials may be used to form the patch support.

Permeability to oxygen may serve as a proxy for permeability to chlorine dioxide. In some embodiments, the patch support will have an oxygen transmission rate (O₂TR) of at least 100 cm³/m²/24 hours at 1 atmosphere and 23° C., such as least 250 cm³/m² per 24 hours at 1 atmosphere and 23° C. Oxygen transmission rate (O₂TR) may be determined by any suitable method. For example, oxygen transmission rate may be determined according to ASTM D398S.

The patch support may be formed from fibers or material in any other suitable form. In some embodiments, the patch support comprises a nonwoven material. In some embodiments, the nonwoven material comprises spun polyolefin fibers, polyesters fibers, polyamide fibers, or the like. In some embodiments, the patch support comprises polyethylene fibers. In some embodiments, the polyethylene fibers are high-density polyethylene fibers. In some embodiments, the high-density polyethylene fibers are flash spun high-density polyethylene fibers. One suitable example of flash spun high-density polypropylene fibers is Dupont's TYVEK® sheet material.

In some embodiments, the patch support is paper or cloth. In some embodiments, the patch support is a polymeric film that is permeable to chlorine dioxide or that is perforated or otherwise modified to be permeable to chlorine dioxide.

Preferably the patch support is opaque to ultraviolet radiation, particularly ultraviolet light having a wavelength in a range from about 200 nm to about 390 nm. For example, the patch support blocks transmission of more than 90% or more of ultraviolet light. In some embodiments, the patch support blocks transmission of 95% or more or ultraviolet light. Having an ultraviolet opaque patch support may be helpful in methods for accelerating the breakdown of chlorine dioxide to reduce the amount of time from activation of chlorite ions to safely opening a package that includes a patch having a sealing layer containing the chlorite ions.

Examples of materials that may be ultraviolet opaque and may be used to form the patch support include polymers with aromatic moieties that absorb UV254 nm light such as polyesters, aromatic polyamides, polystyrene and the like. Flash spun high-density polyethylene fibers, such as Dupont's TYYEK®, may be ultraviolet opaque.

The patches or sheets for forming the patches may include any suitable patch sealing layer comprising chlorite ions. As used herein, a “patch sealing layer” is a layer comprising a composition configured to affix the patch to another structure, such as a surface of a package or packaging film, via fusion bonding or chemical bonding, such as adhesion. The patch sealing layer may be a monolayer or a multilayer. For example, a monolayer patch sealing layer may comprise the chlorite ions mixed with a heat sealable polymeric composition. A multilayer patch sealing layer may comprise a first layer containing chlorite ions and a second separate layer having sealing functionality. For example, second separate layer having sealing functionality of the patch sealing layer may comprise a heat sealable polymeric composition, a cold seal adhesive, or a pressure sensitive adhesive. The patch may be adhered to the packaging film over the entire sealing layer surface, or just a portion of the sealing layer surface (i.e. spot bonded or bonded at an edge of the patch).

The patch sealing layer may be applied to any suitable support to form the patch or the sheet for forming the patch. It will be understood that the patch or the sheet for forming the patch are “films.” As used herein, a “film” is thin structure having a length and width substantially greater than its depth or thickness. Typically, the length and width of a film is at least 100 times greater than the thickness of the film, such as at least 1000 times greater than the thickness of the film. Accordingly, the term “film” may include paper, cloth, non-woven materials, as well as polymeric films.

Typically, the patch sealing layer comprises at least about 0.1% of by weight chlorite salt so that a sufficient amount of chlorine dioxide may be generated. The patch sealing layer may include any suitable amount of chlorite salt. The amount of chlorite salt can be varied to help control the amount of ClO₂ gas is generated. In non-limiting examples, the weight percent of the chlorite salt is, for example, 0.1%, 1%, 5%, 10%, 15%, 20%, 25%, 30%, 35%, 40%, 45%, 50%, 55%, 60%, 65% or 70% of the weight of the patch sealing layer, or any amount in between. In some embodiments, the lower range of the weight of the chlorite salt may be, for example, 0.1%, 1%, 5%, 10%, 15%, 20%, 25%, 30%, 35%, 40%, 45%, 50%, 55%, 60%, or 65% of the weight of the patch sealing layer composition, while the upper range of the weight of the chlorite salt may be 15%, 20%, 25%, 30%, 35%, 40%, 45%, 50%, 55%, 60%, 65%, or 70% of the weight of a patch sealing layer composition. This disclosure encompasses all weight percentage ranges that are defined by any combination of these lower and upper bounds.

The composition of the patch sealing layer is designed to incorporate sufficient sodium chlorite for desired ClO₂ release levels while maintaining a stable dispersion. An example of a coating formulation that meets the purpose comprises sodium chlorite solution; deionized water, a base for the coating formulation (e.g., a polyolefin copolymer dispersion), a viscosity modifier (e.g., xanthan gum), and a defoaming agent (e.g., a non-ion-ionic surfactant).

Regardless of the composition or type of the patch sealing layer and the process for applying the patch sealing layer, the patch sealing layer may have any suitable thickness. In some embodiments, the resulting patch sealing layer or a sheet for forming the patch has a thickness from about 0.1 mils to about 1 mil, such as from about 0.25 mils to about 0.75 mils. When the coating is applied to, for example, non-woven materials the coating may be applied at any suitable weight. For example, the dry coating weight may be from about 1 to about 15 pounds per ream (lb/rm), such as from about 3 to about 11 lb/rm.

Preferably the patch sealing layer is substantially transparent to ultraviolet radiation. The patch sealing layer may be applied to the patch support in any suitable manner. For example, the patch sealing layer and the patch support may be co-extruded to form a film that may form the patch or a sheet for forming the patch. The patch sealing layer may be coated on, sprayed on, rolled on, printed on, adhered to or otherwise applied to the patch support. The patch sealing layer may be disposed across an entire surface of the patch support or across one or more portions of the surface of the patch support. In some embodiments, the patch sealing layer is applied to the entire surface of the patch support using a gravure or air knife coating process.

The patch sealing layer may comprise more than one layer provided that chlorite ions are present in at least one layer. The layer containing the chlorite ions and any layers between the chlorite ion containing layer and the surface of the sealing layer are substantially transparent to ultraviolet radiation.

### Methods of Making and Using Including Gas Generation

In general terms, the packages disclosed herein are made by providing an unsealed package and self-sterilizing components, introducing a gas comprising carbon dioxide to the unsealed package, and hermitically sealing a product in an interior of a self-sterilizing package. Thereafter, the self-sterilizing package is exposed to ultra-violet radiation at which point chlorine dioxide gas generation takes place, allowing for deodorizing, disinfecting, and/or sterilization.

The films described herein may be made in any suitable manner, such as by conventional processes. Processes to produce flexible films may include e.g. cast or blown film extrusion processes, or laminating processes.

Packages may be formed from films in any suitable manner. In some embodiments, the packages are formed by heat sealing a film to itself or another suitable film. In some embodiments, packages such as trays are thermoformed. In some embodiments, films are heat sealed across an opening of a container or base material.

In some embodiments, the package interior is exposed to humidified gas to introduce water. The humidified gas may have any suitable relative humidity. For example, the relative humidity of the humidified gas may be within the range of about 20% to 100%. In some such embodiments, the relative humidity of the humidified gas is within the range of about 20% to 100%. In some such embodiments, the relative humidity of the humidified gas is within the range of about greater than or equal to 60% to less than or equal to 100%.

In some embodiments, a gas flush is used to provide the carbon dioxide to the package interior. The package headspace is flushed with a gas that contains carbon dioxide just prior to sealing the package. The gas flushing may be completed with a gas of 100% carbon dioxide or a gas mixture comprising less than 100% carbon dioxide. In this way, CO₂ is injected into the headspace of a package and then the package is sealed effectively boosting the carbon dioxide content of the headspace to be at least 5%.

The packages described herein may be exposed to UV radiation in any suitable manner to generate chlorine dioxide from the plurality of chlorite ions. The packages are designed to include water in the interior. For example, the package interior may be exposed to water vapor or humidified gas. Upon exposure to UV light, ClO₂ gas is released. Sufficient moisture may be present in the package, for example due to the manufacturing process used to produce the sterile barrier packaging film or the environmental conditions, such that the film or package need only be exposed to UV light to produce ClO₂. In some embodiments, there may be an intervening storage time between creating a self-sterilizing package and its exposure to ultraviolet light by. In some such embodiments, the intervening storage time is within the range of about one minute to about two days. In some such embodiments, the storage time is within the range of about one hour to about one day.

The amount of ClO₂ gas generated from the self-sterilizing components as described herein can be regulated by, for example, varying the wavelength and exposure time of the ultraviolet light, the amount of water vapor (moisture) present, the concentration of chlorite salts in the composition, or the length of the storage period. According to embodiments herein, the amount of ClO₂ gas generated for a given set of UV exposure, water, chlorite concentration and package configuration is increased when CO₂ gas is present as compared with the absence of added CO₂ gas.

In some embodiments, the UV light has a wavelength in the range of about 200 nm to 400 nm. In some such embodiments, the UV light has a wavelength in the range of about 230 nm to 320 nm. In some such embodiments, the UV light has a wavelength in the range of about 240 nm to 280 nm. Preferably, the UV light includes light having a wavelength of 254 nm.

In some embodiments, the package is exposed to UV light for a period of time that is greater than 10 milliseconds. In some embodiments, the package is exposed to UV light for a period of time that is greater than 10 seconds. In some embodiments, the package is exposed to UV light for a period of time that is greater than ten minutes.

In some embodiments, the step of exposing the package to ultraviolet light may be repeated one or more times.

In some embodiments, the method further includes the step of heating the package.

In some embodiments, a method for generating ClO₂ gas includes the steps of (a) providing self-sterilizing components of water and chlorite ions in an interior of a package in an atmosphere of greater than or equal to 5% carbon dioxide, and (b) exposing the package to ultraviolet (UV) light. Optionally, step (b) may be repeated one or more times to generate additional amounts of ClO₂ gas.

A method herein of sterilizing a packaged product comprises: providing an unsealed package comprising: a sterile barrier packaging film comprising a primary layer and a plurality of chlorite ions; and a package interior; and introducing water to the package interior; introducing a product to be sterilized to the package interior, introducing a gas comprising carbon dioxide to the package interior of the package; hermetically sealing an open end of the unsealed package after introducing the gas comprising carbon dioxide to form a self-sterilizing package containing a packaged product and a headspace, wherein the headspace comprises the carbon dioxide in an amount of greater than or equal to 5% by volume of the headspace; and exposing the self-sterilizing package to UV radiation; wherein self-sterilizing components comprise: the water and the plurality of chlorite ions, and are substantially free of an energy-activated catalyst and of an acid-releasing compound. The water is proximate to the plurality of chlorite ions.

Before describing several exemplary embodiments of the invention, it is to be understood that the invention is not limited to the details of construction or process steps set forth in the following description. The invention is capable of other embodiments and of being practiced or being carried out in various ways.

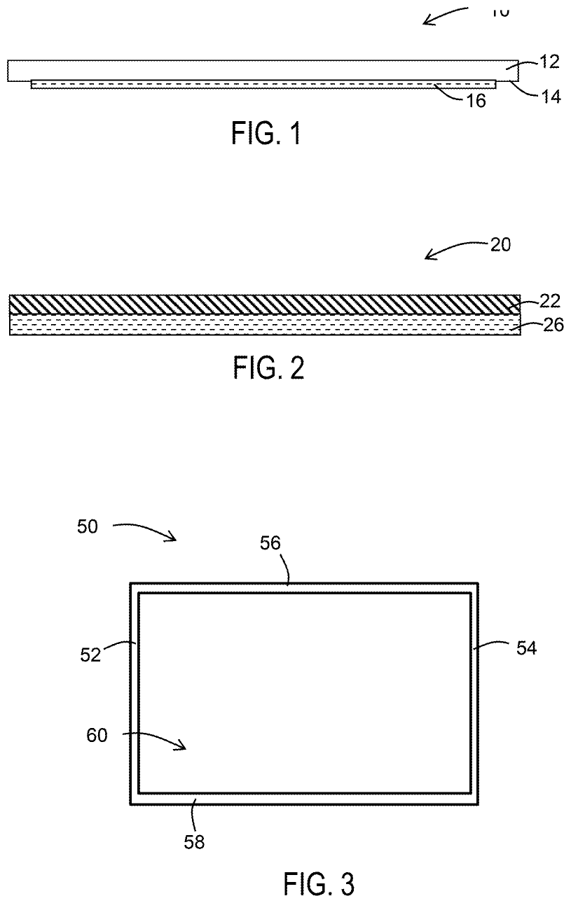

Turning to the figures, FIG. 1 is a schematic side view of a sterile barrier packaging film according to an embodiment. In FIG. 1, the sterile barrier packaging film 10 comprises a primary layer 12 and a coating comprising chlorite ions 16. The primary layer 12 may be a sealing layer having a polymer composition as described herein. The coating may cover the entirety of an inner surface 14 of the primary layer 12, or the coating 16 may cover less than the entirety of the inner surface 14. In one or more embodiments, the coating covers greater than or equal to 10% to less than or equal to 100% of the surface area of the inner surface 14, and all values and subranges therebetween, including greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 95%.

In FIG. 2, which provides a schematic side view of a sterile barrier packaging film according to an embodiment, a sterile barrier packaging film 20 comprises a primary layer 26 and an optional secondary layer 22. The primary layer 26 comprises chlorite ions and a sealing polymer or polymer-based formulation. The optional secondary layer 22 may be an outer layer when present. The primary layer 26 may be applied to the secondary 22 in any suitable manner to form the film. Preferably, the process for applying the primary layer 26 to the secondary layer 22 is performed at temperatures substantially lower that those typically associated with extrusion due to the heat sensitivity of the chlorite ions. Preferably, the primary layer 26 is applied to the secondary layer 22 at room temperature.

FIG. 3 provides a schematic plan view of a package according to an embodiment. Package 50 of FIG. 3 comprises two films that are sealed together as sidewalls, including a first sidewall 60 and a second sidewall (not shown). In this embodiment, the sidewalls are sealed together at first 52, second 54, third 56, and fourth 58 side seams or heat seals, and the volume within the seams or seals at least partially defines an interior of the package. The first sidewall 60 comprises a packaging film according to embodiments herein including but not limited to the films of FIGS. 1 and 2, which comprise chlorite ions. The other sidewall (not shown) may or may not include a multilayer packaging film having a chlorine dioxide-producing layer.

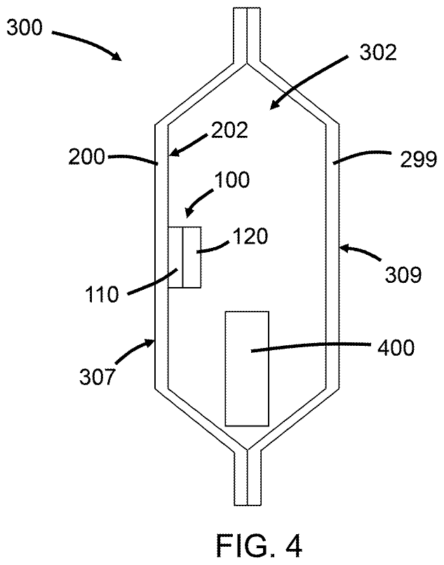

FIG. 4 is a schematic sectional view of a package according to an embodiment. A package 300 is formed from a first packaging film 200 and a second packaging film 299, which may be made of the same or different materials. A patch 100 is affixed to a surface 202 of the first packaging film 200 that defines at least a portion of the package interior 302 having an interior volume. The patch 100 comprises a patch support 120 and a patch sealing layer 110 comprising chlorite ions. The patch 100 is affixed to the surface 202 of the first packaging film 200 via the patch sealing layer 110. The patch support 120 is permeable to chlorine dioxide and is preferably opaque to ultraviolet light. The patch sealing layer 110 comprising chlorite ions is facing the exterior of the package so that when UV light is applied to the package, the chlorite ions are readily available for generating ClO₂ gas. An added benefit is that the chlorite salt containing sealing layer is not in direct contact with a product in the package.

The first 200 and second 299 packaging films are substantially transparent to ultraviolet light and are substantially impermeable to chlorine dioxide. The first packaging film 200 defines a first side 307 of the package 300, and the second packaging film 299 defines a second side 309 of the package 300.

An article or product 400 is disposed in the package interior 302. Ultraviolet light may be applied to the package 300 through the first side 307 to generate chlorine dioxide gas from chlorite ions in the patch sealing layer 110. The generated-chlorine dioxide may deodorize, disinfect, or sterilize the article or product 400.

### EXAMPLES

Patch. Patches used in the examples were made as follows. The patches comprised a patch support permeable to chlorine dioxide and a sealing layer. The patch support was TYVEK 1073B coated with the coating formulation of Table 1, which made the sealing layer.

TABLE 1

| Material | Function | wt. % |
|---|---|---|
| Polyolefin copolymer dispersion | Base | 44.32 |
| Dow HYPOD ™ 8503 | | |
| Water | — | 38.66 |
| Xanthan Gum | viscosity modifier | 0.08 |
| Keltrol AP | | |
| Sodium chlorite solution | Source of chlorite ions | 16.91 |
| Adox BCD-25 from. International | | |
| Dioxide | | |
| Non-ionic surfactant | defoaming agent | 0.03 |
| Air Products Surfynol 107L | | |

Each patch had a coat-weight of about 7 pounds per ream (lb/rm).

In the Examples, when pouches with patches were exposed to ultraviolet light having a wavelength of 254 nm (UV254) for 60 seconds, exposure to UV254 was conducted using an XL-1500 UV crosslinker unit (contains six 16W UV254 bulbs) at a distance of 2″ from the UV bulbs.

Adapted Palintest. In the Examples, when extracted gas was analyzed for ClO₂ gas concentration, a Palintest adapted for packaging headspace analysis purposes was used (herein referred to as the “Adapted Palintest”), which was conducted as follows for each pouch having a headspace. The measurements were conducted in a fume hood and each pouch was agitated to ensure even concentration prior to testing. A syringe is used to puncture one side of the pouch to extract 20 mL of the headspace gas. The syringe needle is immediately placed (to prevent leakage) into 40 mL of chilled DI water (38° F.) in a beaker to aspirate 5 mL of the chilled water. The syringe is then shaken vigorously to dissolve the ClO₂ gas into the water. The 5 mL of water with dissolved ClO₂ gas is then expelled into the beaker. Another flush of the syringe is conducted by aspirating 5 mL of the beaker contents into the syringe, shaking the syringe, and then expelling the syringe contents into the beaker. The contents of the beaker of ClO₂ dissolved in water is then analyzed by a Palintest ChlordioX Plus to determine ClO₂ content (mg). Based on a standard of 40 mL chilled DI water and 20 mL headspace gas, in order to calculate the concentration of ClO₂, converting units from mg ClO₂/L water to mg ClO₂/L headspace is done by multiplying the ClO₂ content (mg) by 0.04 L water and divide by 0.02 L headspace. A shortcut calculation based on the standard can be performed by simply multiplying the Palintest content by 2 to get the headspace concentration.

### Comparative Example 1

### Example 2

### Comparative Example 3

Pouches with a patch. In Examples 1-3, pouches with a patch for delivering chlorite ions were made as follows. A sterile barrier packaging film comprising a layer of biaxially-oriented polyamide (BOPA) and a multilayer film containing a polyethylene (PE)-based layer was obtained. The PE-based layer served as a heat seal layer for forming the pouch. The patches made as described above were applied to the PE-based layer.

Two pieces of the sterile barrier packaging film were cut to 8″×10″ in size (BOPA/PE), and 1 patch (4 inches (10.16 cm) diameter) was sealed to one of the PE-based layers with the patch sealing layer facing the exterior of the PE-based layer. The two pieces of sterile barrier packaging film were then heat sealed together such that the PE-based layers were facing each other, and the patch sealed to one of the films was now between the films. The films were sealed on three sides to form seams, resulting in an unsealed pouch with an open fourth side.

The unsealed pouches were then exposed to a moisturization step by their storage for 2 hours in a room having a temperature of 35° C. and 80% relative humidity (RH).

The unsealed pouches were then exposed to vacuum and sealed on the fourth side. A test gas of either air or carbon dioxide was then injected with a gas flush into each of the pouches to create a headspace and then resealed by heat seal.

A first set of comparative pouches according to COMPARATIVE EXAMPLE 1 (Samples 1.1-1.3) received 40 mL of air in the headspace. A second set of inventive pouches according to EXAMPLE 2 (Samples 2.1-2.3) received 40 mL of CO₂. A third set of comparative pouches according to COMPARATIVE EXAMPLE 3 (Samples 3.1-3.3) received 40 mL of CO₂.

COMPARATIVE EXAMPLE 1 and EXAMPLE 2 were exposed to ultraviolet light having a wavelength of 254 nm (UV254) for 60 seconds. COMPARATIVE EXAMPLE 3 was not exposed to UV254.

All of COMPARATIVE EXAMPLE 1, EXAMPLE 2, and COMPARATIVE EXAMPLE 3 were placed in a completely dark drawer having no ambient light for 5 minutes. The use of a completely dark drawer is to avoid degradation of the CO₂ gas by ambient light. Thereafter in a fume hood, 20 mL of headspace gas was extracted and dissolved in 40 mL of chilled deionized water. The extracted gas was analyzed for ClO₂ concentration using the Adapted Palintest.

Table 2 summarizes the samples and ClO₂ concentration measurements.

TABLE 2

| | | | |
|---|---|---|---|
| | | | Extracted Gas |
| | | | ClO₂ |
| | Gas in | Exposure | Concentration |
| SAMPLE | Headspace | to UV? | (mg/L) |

COMPARATIVE EXAMPLE 1

| | | | |
|---|---|---|---|
| 1.1 | Air | Yes | 7.08 |
| 1.2 | Air | Yes | 7.10 |
| 1.3 | Air | Yes | 7.76 |

EXAMPLE 2

| | | | |
|---|---|---|---|
| 2.1 | CO₂ | Yes | 16.36 |
| 2.2 | CO₂ | Yes | 14.70 |
| 2.3 | CO₂ | Yes | 15.62 |

COMPARATIVE EXAMPLE 3

| | | | |
|---|---|---|---|
| 3.1 | CO₂ | No | <0.02* |
| 3.2 | CO₂ | No | <0.02* |
| 3.3 | CO₂ | No | <0.02* |

*a measurement of <0.02 mg/L is the lowest measurement possible on the test device.

From Table 2, it is concluded that exposure of a chlorite-containing patch to UV254 for 60 seconds in a headspace of 100% by volume CO₂ generates about twice as much ClO₂ as a headspace of air. In the absence of exposure to UV254 and a headspace of 100% by volume CO₂, ClO₂ was not detectable indicating that carbonic acid was not spontaneously reacting with the sodium chlorite.

### Example 4

### Comparative Example 5

Pouches with a patch. In Examples 4-5, pouches with a patch for delivering chlorite ions were made as follows. A sterile barrier packaging film comprising a layer of biaxially-oriented polyamide (BOPA) and a multilayer film containing a polyethylene (PE)-based layer was obtained. The PE-based layer served as a heat seal layer for forming the pouch. The patches made as described above were applied to the PE-based layer with the patch sealing layer facing the exterior of the package.

Two pieces of the sterile barrier packaging film were cut to 8″×10″ in size (BOPA/PE), and 1 patch (4 inches (10.16 cm) diameter) was sealed to one of the PE-based layers with the patch sealing layer facing the exterior of the PE-based layer. The two pieces of sterile barrier packaging film were then heat sealed together such that the PE-based layers were facing each other, and the patch sealed to one of the films was now between the films. The films were sealed on three sides to form seams, resulting in an unsealed pouch with an open fourth side.

The unsealed pouches were then exposed to a moisturization step by exposure to a handheld steamer 10 seconds from a distance of about 5 inches from the open fourth side.

The unsealed pouches were then exposed to vacuum and sealed on the fourth side. A volume of 100 mL test gas was then injected into each of the pouches to create a headspace. Composition of the test gas varied in CO₂ content.

A first set of pouches according to EXAMPLE 4 (Samples 4.1-4.5) received CO₂-containing gas varying from 20% by volume to 100% by volume. A comparative pouch according to COMPARATIVE EXAMPLE 5 (Sample 5.1.) received 100% air.

EXAMPLE 4 and COMPARATIVE EXAMPLE 5 were exposed to ultraviolet light having a wavelength of 254 nm (UV254) for 60 seconds.

EXAMPLE 4 and COMPARATIVE EXAMPLE 5 were placed in a completely dark drawer having no ambient light for 5 minutes. The use of a completely dark drawer is to avoid degradation of the ClO₂ gas by ambient light. Thereafter in a fume hood, 20 mL of headspace gas was extracted and dissolved in 40 mL of chilled deionized water. The extracted gas was analyzed for ClO₂ concentration using the Adapted Palintest.

Table 3 summarizes the samples and ClO₂ concentration measurements.

TABLE 3

| | | Extracted Gas |
|---|---|---|
| | Headspace | ClO₂ |
| | Composition | Concentration |
| SAMPLE | (volume %) | (mg/L) |
| EXAMPLE 4 | | |
| 4.1 | 100% CO₂ | 11.94 |
| 4.2 | 80% CO₂/20% Air | 10.54 |
| 4.3 | 60% CO₂/40% Air | 10.60 |
| 4.4 | 40% CO₂/60% Air | 11.62 |
| 4.5 | 20% CO₂/80% Air | 11.50 |
| COMPARATIVE EXAMPLE 5 | | |
| 2.1 | 100% Air | 6.10 |

From Table 3, it is concluded that exposure of a chlorite-containing patch to UV254 in a headspace of 100% by volume CO₂ generated about 2 times as much ClO₂ as a headspace of 100% air. CO₂ content varying from 20% by volume to 80% by volume also showed increased ClO₂ relative to a headspace of 100% air.

### Example 6

### Comparative Example 7

In Examples 6-7, pouches with a patch for delivering chlorite ions were made according to Examples 4-5.

A first set of pouches according to EXAMPLE 6 (Samples 6.1-4.4) received CO₂-containing gas varying from 5% by volume to 100% by volume. A comparative pouch according to COMPARATIVE EXAMPLE 7 (Sample 7.1) received 100% air.

EXAMPLE 6 and COMPARATIVE EXAMPLE 7 were exposed to ultraviolet light having a wavelength of 254 nm (UV254) for 60 seconds.

EXAMPLE 6 and COMPARATIVE EXAMPLE 7 were placed in a completely dark drawer having no ambient light for 5 minutes. The use of a completely dark drawer is to avoid degradation of the ClO₂ gas by ambient light. Thereafter in a fume hood, 20 mL of headspace gas was extracted and dissolved in 40 mL of chilled deionized water. The extracted gas was analyzed for ClO₂ concentration using the Adapted Palintest.

Table 4 summarizes the samples and ClO₂ concentration measurements.

TABLE 4

| | | Extracted Gas |
|---|---|---|
| | Headspace | ClO₂ |
| | Composition | Concentration |
| SAMPLE | (volume %) | (mg/L) |
| EXAMPLE 6 | | |
| 6.1 | 100% CO₂ | 11.32 |
| 6.2 | 15% CO₂/85% Air | 10.68 |
| 6.3 | 10% CO₂/90% Air | 9.12 |
| 6.4 | 5% CO₂/95% Air | 8.98 |
| COMPARATIVE EXAMPLE 7 | | |
| 7.1 | 100% Air | 5.64 |

From Table 4, it is concluded that exposure of a chlorite-containing patch to UV254 in a headspace of down to 5% by volume CO₂ generated about 1.5 times as much ClO₂ as a headspace of 100% air. CO₂ contents of 10% by volume, 15% by volume, and 100% by volume also showed increased ClO₂ relative to a headspace of 100% air.

### Example 8

### Comparative Examples 9-12

Pouches with chlorite ions. In Examples 8-12, pouches for delivering chlorite ions were made as follows.

A 3-sided sealed pouch was made on a production packaging machine with dimensions of 5″×10.5″. A 4″ in diameter Tyvek patch coated with the coating in Table 1 was sealed to the inside.

The pouches with an unsealed fourth side were then exposed to a moisturization step by their storage for 2 hours in a room having a temperature of 35° C. and 80% relative humidity (RH).

The unsealed pouches were then exposed to vacuum and sealed on the fourth side. A test gas was then injected by a gas flush into each of the pouches to create a headspace. Various test gases were analyzed.

Each set of pouches received 40 mL of test gas in the headspace as shown in Table 5.

All samples of the Examples 8-12 were exposed to ultraviolet light having a wavelength of 254 nm (UV254) for 60 seconds and then were placed in a completely dark drawer having no ambient light for 5 minutes. The use of a completely dark drawer is to avoid degradation of the ClO₂ gas by ambient light. Thereafter in a fume hood, 20 mL of headspace gas was extracted and dissolved in 40 mL of chilled deionized water. The extracted gas was analyzed for ClO₂ concentration using the Adapted Palintest.

Table 5 summarizes the samples and the ClO₂ concentration measurements.

TABLE 5

| | | Extracted Gas |
|---|---|---|
| | | ClO₂ |
| | | Concentration |
| SAMPLE | Headspace Gas | (mg/L) |
| EXAMPLE 8 | | |
| 8.1 | CO₂ | 16.36 |
| 8.2 | CO₂ | 14.70 |
| 8.3 | CO2 | 15.62 |
| COMPARATIVE EXAMPLE 9 | | |
| 9.1 | Air | 7.08 |
| 9.2 | Air | 7.10 |
| 9.3 | Air | 7.76 |
| COMPARATIVE EXAMPLE 10 | | |
| 10.1 | N₂ | 5.70 |
| 10.2 | N₂ | 6.56 |
| 10.3 | N₂ | 6.70 |
| COMPARATIVE EXAMPLE 11 | | |
| 11.1 | O₂ | 7.70 |
| 11.2 | O₂ | 7.76 |
| 11.3 | O₂ | 7.10 |
| COMPARATIVE EXAMPLE 12 | | |
| 12.1 | He | 7.04 |
| 12.2 | He | 6.52 |
| 12.3 | He | 7.14 |

From Table 5, it is concluded that the presence of CO₂ in the headspace has the greatest effect on increasing ClO₂ concentration relative to the other gases tested: air, N₂, O₂, and He.

### Example 13

### Comparative Example 14

Pouches with chlorite ions. In Examples 13-14, pouches for delivering chlorite ions were made as follows.

Two pieces of a nylon lamination film were cut to 5″×11″. The two pieces were then heat sealed on three sides to form an unsealed pouch with an open fourth side.

A stock 32% by weight sodium chlorite solution was obtained. To each pouch, 3 drops of the chlorite solution were added at the open fourth side using a pipet. No additional moisturization was needed because the sodium chlorite solution contained water.

The unsealed pouches were sealed on the fourth side and a septum was used to remove air from the headspace. A separate pouch filled with 100% CO₂ from a pressurized cylinder, was then used as a reservoir to fill headspaces of the sample pouches.

Each pouch received 40 mL of headspace of a gas as shown in Table 6.

All samples of the Examples 13-14 were exposed to ultraviolet light having a wavelength of 254 nm (UV254) for 75 seconds and then were placed in a completely dark drawer having no ambient light for 5 minutes. The use of a completely dark drawer is to avoid degradation of the ClO₂ gas by ambient light. Thereafter in a fume hood, 20 mL of headspace gas was extracted and dissolved in 40 mL of chilled deionized water. The extracted gas was analyzed for ClO₂ concentration using the Adapted Palintest.

Table 6 summarizes the samples and ClO₂ concentration measurements.

TABLE 6

| | | | Extracted Gas |
|---|---|---|---|
| | | Mass of | ClO₂ |
| | | NaClO₂ | Concentration |
| SAMPLE | Headspace Gas | added (g) | (mg/L) |
| EXAMPLE 13 | | | |
| 13.1 | CO₂ | ~0.032 | 14.3 |
| 13.2 | CO₂ | ~0.032 | 13.2 |
| 13.3 | CO₂ | ~0.032 | 14.0 |
| COMPARATIVE EXAMPLE 14 | | | |
| 14.1 | Air | ~0.032 | 2.44 |
| 14.2 | Air | ~0.032 | 1.75 |
| 14.3 | Air | ~0.032 | 2.21 |

From Table 6, it is concluded that exposure of chlorite ions to UV254 for 75 seconds in a headspace of 100% by volume CO, generates more than 5 times as much ClO₂ as a headspace of air.

### Example 15

### Comparative Example 16

Pouches with a patch. In Examples 13-14, pouches with a patch for delivering chlorite ions were made as follows. A sterile barrier packaging film comprising a layer of nylon and peelable sealant layer was obtained. The peelable sealant layer served as a heat seal layer for forming the pouch. The patches made as described above were applied to the peelable sealant layer with the patch sealing layer facing the exterior.

Two pieces of the sterile barrier packaging film were cut to 8″×8″ (20.32 cm×20.32 cm) in size, and 1 patch (6 inches (15.24 cm))×6 inches (15.24 cm)) was sealed to one of the peelable sealant layers. The two pieces of sterile barrier packaging were then heat sealed on three sides to form an unsealed pouch with an open fourth side.

The unsealed pouches were then exposed to a moisturization step by exposure to a handheld steamer 10 seconds from a distance of about 5 inches from the open fourth side.

The unsealed pouches were then exposed to a vacuum and sealed on the fourth side. A test gas of either air or carbon dioxide was then injected into each of the pouches to create a headspace.

Each pouch received 60 mL of headspace of a gas as shown in Table 7.

Examples 15-16 were exposed to ultraviolet light having a wavelength of 254 nm (UV254) for 60 seconds and then were placed in a completely dark drawer having no ambient light for 5 minutes. The use of a completely dark drawer is to avoid degradation of the ClO₂ gas by ambient light. Thereafter in a fume hood, 20 mL of headspace gas was extracted and dissolved in 40 mL of chilled deionized water. The extracted gas was analyzed for ClO₂ concentration using the Adapted Palintest.

Table 7 summarizes the samples and ClO₂ concentration measurements.

TABLE 7

| | | Extracted Gas |
|---|---|---|
| | | ClO₂ |
| | Gas in | Concentration |
| SAMPLE | Headspace | (mg/L) |
| EXAMPLE 15 | | |
| 15.1 | CO₂ | 23.9 |
| COMPARATIVE EXAMPLE 16 | | |
| 16.1 | Air | 8.51 |

From Table 7, it is concluded that exposure of a chlorite-containing patch to UV254 for 60 seconds in a headspace of 100% by volume CO₂ generates about 2.8 times as much ClO₂ as a headspace of air. It is noted that a patch with more surface area (26 in² in this example vs 12.56 in² in previous examples) generates more ClO₂.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

### Embodiments

Embodiment A: A package comprising:
   a sterile barrier packaging film;
   self-sterilizing components comprising: a plurality of chlorite ions and water, wherein the self-sterilizing components are substantially free of an energy-activated catalyst and of an acid-releasing compound;
   a package interior formed by hermetically sealing the sterile barrier packaging film; and
   a headspace within the package interior comprising carbon dioxide present in an amount of greater than or equal to 5% by volume of the headspace.
Embodiment B: The package of any of Embodiments A or C-L, wherein when the package is exposed to ultraviolet (UV) light having a wavelength of 254 nm, reaction of the chlorite ions with the water generates chlorine dioxide (ClO₂), which is released into the headspace.
Embodiment C: The package of any of Embodiments A-B or D-L, wherein in the absence of any UV light, there is not chlorine dioxide (ClO₂) generation.
Embodiment D: The package of any of Embodiments A-C or E-L, wherein the water is proximate to the plurality of chlorite ions.
Embodiment E: The package of any of Embodiments A-D or F-L having a relative humidity in the package interior in the range of greater than or equal to 20% to less than or equal to 100% at 72.5° F.
Embodiment F: The package of any of Embodiments A-E or G-L, wherein a salt comprising sodium chlorite, potassium chlorite, calcium chlorite, magnesium chlorite, lithium chlorite, ammonium chlorite, or mixtures thereof supplies the plurality of chlorite ions.
Embodiment G: The package of any of Embodiments A-F or H-L, wherein the sterile barrier packaging film is substantially ultraviolet (UV)-light transparent.
Embodiment H: The package of any of Embodiments A-G, wherein the sterile barrier packaging film comprises a primary layer and a patch affixed to the primary layer, wherein the patch supplies the plurality of chlorite ions.
Embodiment I: The package of Embodiment H, wherein the patch comprises:
   a patch support that is permeable to chlorine dioxide and that defines a first major surface of the patch; and
   a patch sealing layer comprising the plurality of chlorite ions dispersed in a polymer composition, wherein the patch sealing layer is in contact with the patch support and defines a second major surface of the patch, and wherein the patch sealing layer is affixed to the primary layer.
Embodiment J: The package of Embodiment I, wherein the polymer composition comprises at least one of: a polyethylene, a polypropylene, a polyacrylate, or a copolymer of any of these.
Embodiment K: The package of any of Embodiments A-G, wherein the plurality of chlorite ions is present in a coating on of the self-sterilizing packaging film, and the coating comprises a chlorite salt in combination with a polymer composition.
Embodiment L: The package of Embodiment K, wherein the polymer composition comprises at least one of: a polyethylene, a polypropylene, a polyacrylate, or a copolymer of any of these.
Embodiment M: A packaged product comprising:
   a packaging film;
   a plurality of chlorite ions;
   water;
   a package interior formed by hermetically sealing the packaging film;
   a product within the package interior; and
   a headspace within the package interior comprising carbon dioxide present in an amount of greater than or equal to 5% by volume of the headspace;
   wherein self-sterilizing components comprise: the water and the plurality of chlorite ions, and are substantially free of energy-activated catalyst and are substantially free of an acid releasing compound.
Embodiment N: The packaged product of any of Embodiments M or O-S, wherein when the packaging film is exposed to ultraviolet (UV) light having a wavelength of 254 nm, reaction of the chlorite ions with the water generates chlorine dioxide (ClO₂), which is released into the headspace and is effective to sterilize the product within the package interior.
Embodiment O: The packaged product of any of Embodiments M, N, R or S, wherein the packaging film comprises a primary layer and a patch affixed to the primary layer, wherein the patch supplies the plurality of chlorite ions.
Embodiment P: The packaged product of Embodiment O, wherein the patch comprises:
   a patch support that is permeable to chlorine dioxide; and
   a patch sealing layer comprising the plurality of chlorite ions dispersed in a polymer composition, wherein the patch sealing layer is in contact with the patch support, and wherein the patch sealing layer is affixed to the primary layer.
Embodiment Q: The packaged product of Embodiment P, wherein the polymer composition comprises at least one of: a polyethylene, a polypropylene, a polyacrylate, or a copolymer of any of these.
Embodiment R: The packaged product of any of Embodiments M-Q or S, wherein the product within the package interior is a medical device or a medical supply.
Embodiment S: The packaged product of any of Embodiments M-R, wherein the product within the package interior is a food.
Embodiment T: A method of sterilizing a packaged product comprising:
   providing an unsealed package comprising:
      a sterile harrier packaging film comprising a primary layer and a plurality of chlorite ions; and
      a package interior; and
   introducing water to the package interior;
   introducing a product to be sterilized to the package interior,
   introducing a gas comprising carbon dioxide to the package interior of the package;
   hermetically sealing an open end of the unsealed package after introducing the gas comprising carbon dioxide to form a self-sterilizing package containing a packaged product and a headspace, wherein the headspace comprises the carbon dioxide in an amount of greater than or equal to 5% by volume of the headspace; and
   exposing the self-sterilizing package to UV radiation;
   wherein self-sterilizing components comprise: the water and the plurality of chlorite ions, and are substantially free of an energy-activated catalyst and of an acid-releasing compound.
Embodiment U: The method of any of Embodiments T, V or W, wherein the water is proximate to the plurality of chlorite ions.
Embodiment V: The method of any of Embodiments T, U or W, wherein an amount of at least 1.39 micrograms/milliliter of chlorine dioxide is generated within the package within a timeframe of 5 minutes.
Embodiment W: The method of any of Embodiments T, U or V, wherein the UV radiation includes a 254 nm wavelength.

1. A package comprising:
a sterile barrier packaging film;
self-sterilizing components comprising: a plurality of chlorite ions and water, wherein the self-sterilizing components are substantially free of an energy-activated catalyst and of an acid-releasing compound;
a package interior formed by hermetically sealing the sterile barrier packaging film; and
a headspace within the package interior comprising carbon dioxide present in an amount of greater than or equal to 5% by volume of the headspace.

2. The package of claim 1, wherein when the package is exposed to ultraviolet (UV) light having a wavelength of 254 nm, reaction of the chlorite ions with the water generates chlorine dioxide (ClO₂), which is released into the headspace.

3. The package of claim 1, wherein in the absence of any UV light, there is not chlorine dioxide (ClO₂) generation.

4. The package of claim 1, wherein the water is proximate to the plurality of chlorite ions.

5. The package of claim 1 having a relative humidity in the package interior in the range of greater than or equal to 20% to less than or equal to 100% at 72.5° F.

6. The package of claim 1, wherein a salt comprising sodium chlorite, potassium chlorite, calcium chlorite, magnesium chlorite, lithium chlorite, ammonium chlorite, or mixtures thereof supplies the plurality of chlorite ions.

7. The package of claim 1, wherein the sterile barrier packaging film is substantially ultraviolet (UV)-light transparent.

8. The package of claim 1, wherein the sterile barrier packaging film comprises a primary layer and a patch affixed to the primary layer, wherein the patch supplies the plurality of chlorite ions.

9. The package of claim 8, wherein the patch comprises:
a patch support that is permeable to chlorine dioxide and that defines a first major surface of the patch; and
a patch sealing layer comprising the plurality of chlorite ions dispersed in a polymer composition, wherein the patch sealing layer is in contact with the patch support and defines a second major surface of the patch, and wherein the patch sealing layer is affixed to the primary layer.

10. The package of claim 9, wherein the polymer composition comprises at least one of: a polyethylene, a polypropylene, a polyacrylate, or a copolymer of any of these.

11. The package of claim 1, wherein the plurality of chlorite ions is present in a coating on of the self-sterilizing packaging film, and the coating comprises a chlorite salt in combination with a polymer composition.

12. The package of claim 11, wherein the polymer composition comprises at least one of: a polyethylene, a polypropylene, a polyacrylate, or a copolymer of any of these.

13. A packaged product comprising:
a packaging film;
a plurality of chlorite ions;
water;
a package interior formed by hermetically sealing the packaging film;
a product within the package interior; and
a headspace within the package interior comprising carbon dioxide present in an amount of greater than or equal to 5% by volume of the headspace;
wherein self-sterilizing components comprise: the water and the plurality of chlorite ions, and are substantially free of energy-activated catalyst and are substantially free of an acid releasing compound.

14. The packaged product of claim 13, wherein when the packaging film is exposed to ultraviolet (UV) light having a wavelength of 254 nm, reaction of the chlorite ions with the water generates chlorine dioxide (ClO₂), which is released into the headspace and is effective to sterilize the product within the package interior.

15. The packaged product of claim 13, wherein the packaging film comprises a primary layer and a patch affixed to the primary layer, wherein the patch supplies the plurality of chlorite ions.

16. The packaged product of claim 15, wherein the patch comprises:
a patch support that is permeable to chlorine dioxide; and
a patch sealing layer comprising the plurality of chlorite ions dispersed in a polymer composition, wherein the patch sealing layer is in contact with the patch support, and wherein the patch sealing layer is affixed to the primary layer.

17. The packaged product of claim 16, wherein the polymer composition comprises at least one of: a polyethylene, a polypropylene, a polyacrylate, or a copolymer of any of these.

18. The packaged product of claim 13, wherein the product within the package interior is a medical device or a medical supply.

19. The packaged product of claim 13, wherein the product within the package interior is a food.

20. (canceled)

21. (canceled)

22. (canceled)

23. (canceled)
